(19) United States
(12) Patent Application Publication
(10) Pub. No.: US 2015/0119317 A1
(43) Pub. Date: Apr. 30, 2015

(54) ORAL SOLID DOSAGE FORMULATION OF 1,1-DIMETHYLETHYL [(1S)-1-CARBAMOYL)PYRROLIDIN-1-YL]CARBONYL}-2,2-DIMETHYLPROPYL]CARBAMATE

(71) Applicant: BRISTOL-MYERS SQUIBB COMPANY, Princeton, NJ (US)

(72) Inventors: Rhye Hamey, Newtown, PA (US); Preetanshu Pandey, Piscataway, NJ (US); Dilbir S. Bindra, Hillsborough, NJ (US); Chandra Vemavarapu, Hillsborough, NJ (US); Robert Kevin Perrone, Belle Mead, NJ (US)

(21) Appl. No.: 14/399,114

(22) PCT Filed: May 3, 2013
(86) PCT No.: PCT/US2013/039378

§ 371 Date: Nov. 5, 2014

Related U.S. Application Data
(60) Provisional application No. 61/643,486, filed on May 7, 2012.

(51) Int. Cl. A61K 38/06; A61K 31/355

(52) U.S. Cl. CPC A61K 38/06; A61K 31/355
USPC 514/3.8; 514/21.9

(57) ABSTRACT

The present invention is directed to an oral solid dosage formulation of Asunaprevir, 1,1-dimethylethyl[(1S)-1-{[(2S,4R)-4-(7-chloro-4methoxyisoquinolin-1-yloxy)-2-({(1R,2S)-1-[(cyclopropylsulfonyl)carbamoyl]-2-ethenylcyclopropyl}carbamoyl)pyrrolidin-1-yl]carbonyl}-2,2-dimethylpropyl]carbamate, and to methods of using the formulation in the treatment and/or inhibition of the hepatitis C virus and infections caused thereby.

### FIELD OF THE INVENTION

The invention relates generally to an oral solid dosage formulation for poorly water soluble pharmaceutical compounds that exhibit significant food effect on oral bioavailability. In particular, the invention relates to a new oral solid dosage formulation of Asunaprevir, 1,1-dimethylethyl[(1S)-1-{[(2S,4R)-4-(7-chloro-4methoxyisoquinolin-1-yloxy)-2-({(1R,2S)-1-[(cyclopropylsulfonyl)carbamoyl]-2-ethenylcyclopropyl}carbamoyl)pyrrolidin-1-yl]carbonyl}-2,2-dimethylpropyl]carbamate, to methods of administering the formulation comprising the compound to provide a total blood plasma concentration profile that is greater than the total blood plasma concentration of an orally administered solution comprising the compound, and to methods of using the formulation for the treatment and/or inhibition of the hepatitis C virus and infections caused thereby.

### BACKGROUND OF THE INVENTION

The compound of formula (I), Asunaprevir, 1,1-dimethylethyl[(1S)-1-{[(2S,4R)-4-(7-chloro-4methoxyisoquinolin-1-yloxy)-2-({(1R,2S)-1-[(cyclopropylsulfonyl)carbamoyl]-2-ethenylcyclopropyl}carbamoyl)pyrrolidin-1-yl]carbonyl}-2,2-dimethylpropyl]carbamate, is a selective NS3 protease inhibitor and is useful in the treatment of the hepatitis C virus (HCV)

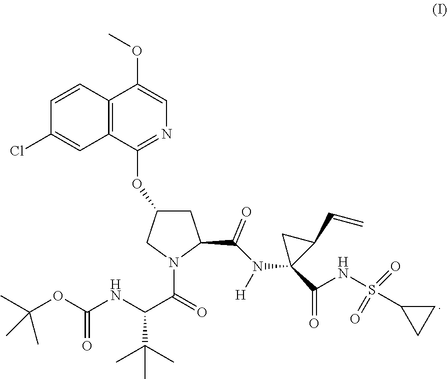 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

The compound of formula (I) and its preparation has been previously described in U.S. Pat. No. 6,995,174, issued Feb. 7, 2006, U.S. Pat. No. 7,449,479, issued Nov. 11, 1988, and U.S. Pat. No. 7,915,291 which issued Mar. 29, 2011.

Asunaprevir is a poorly water soluble compound that in various formulations has exhibited a significant food effect. The present invention provides an oral solid dosage formulation that can be manufactured through conventional wet granulation technology, is amenable to fixed dose combinations, mitigates the food effect, and increases the bioavailability of the compound in the fasted state.

Clinical studies of Asunaprevir have shown that the current formulation needs to be improved due to both low bioavailability and food effects observed in the trials. It has surprisingly been found that an oral solid dosage formulation employing d-alpha tocopheryl polyethylene glycol 1000 succinate (Vitamin ETPGS) results in enhanced bioavailability as well as mitigation of the food effects observed in the current clinical formulation as well as other proposed formulations.

Vitamin E TPGS is well known as a bioavailability enhancer. Due to its low melting point, there is limited publication with it being used in solid dosage formulations. For this formulation, Vitamin E TPGS is used as a binder, surfactant, solubilizer, lubricant, and bioavailability enhancer. While not surmising the mechanism involved, it was surprisingly and unexpectedly found that when Vitamin E TPGS is combined with the other surfactant(s) used in the formulation, such as poloxamer and sodium lauryl sulfate, that the formulation was able to mitigate the food effect observed with other formulations. Vitamin E TPGS administered by itself or in combination with other surfactants is not known to mitigate a food effect.

Thus, it was observed that using only about 4-5% Vitamin E TPGS resulted in a threefold increase in bioavailability as well as significant mitigation of the food effect.

Thus, it is an object of the invention to prepare oral solid dosage formulations that will increase the bioavailability of an API with low solubility, such as Asunaprevir, and mitigate any observed food effects.

### SUMMARY OF THE INVENTION

The present invention is directed to an oral solid dosage formulation of the compound of formula (I), to methods of administering the formulation comprising the compound to provide a total blood plasma concentration profile that is greater than the total blood plasma concentration of an orally administered solution comprising the compound, and to methods of treating and/or inhibiting the hepatitis C virus using said formulation.

### DETAILED DESCRIPTION OF THE INVENTION

In a first aspect, the present invention is directed to an oral solid dosage formulation of the compound I of the formula

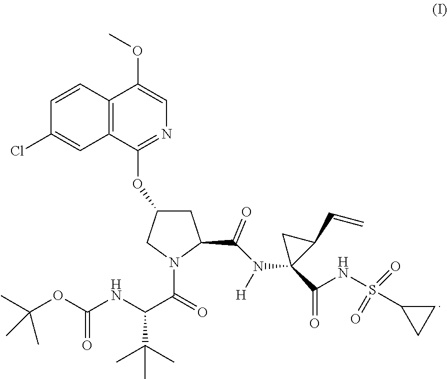 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

In a second aspect of the invention, there is disclosed an oral solid dosage formulation comprising at least one pharmaceutical agent comprising Compound I of the formula

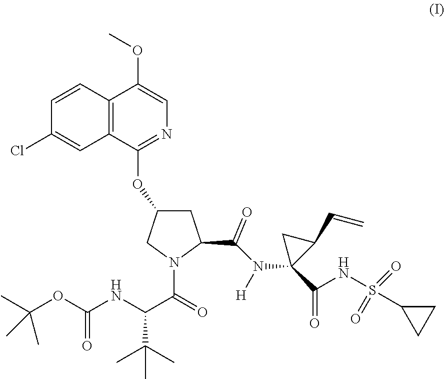 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

in the range of 30-80% w/w and a bioavailability enhancer is included in the range of 2-20% w/w of the total formulation.

In an embodiment of the second aspect of the invention, there is disclosed the oral solid dosage formulation wherein a surfactant is included in the range of 2-10%.

In another embodiment of the second aspect of the invention, there is disclosed the oral solid dosage formulation wherein the active pharmaceutical agent is included in the formulation in an amount of at least about 40% w/w.

In another embodiment of the second aspect of the invention, there is disclosed the oral solid dosage formulation wherein the active pharmaceutical agent is included in the formulation in an amount of at least about 50% w/w.

In another embodiment of the second aspect of the invention, there is disclosed the oral solid dosage formulation wherein the active pharmaceutical agent is included in the formulation in an amount of at least about 60% w/w.

In another embodiment of the second aspect of the invention, there is disclosed the oral solid dosage formulation wherein the formulation is a tablet.

In another embodiment of the second aspect of the invention, there is disclosed the oral solid dosage formulation wherein the formulation is a wet granulated tablet.

In a third aspect of the invention, there is disclosed a method of administering an oral solid dosage formulation comprising orally administering to a fasted mammalian subject the formulation comprising Compound I having the formula

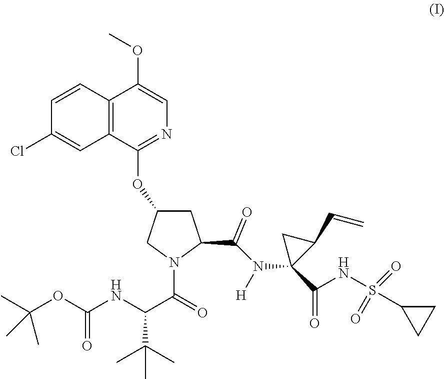 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

to provide a blood plasma concentration profile after an initial dose of the formulation with a Cmax of Compound I after an initial dose of the formulation that is at least greater than about 25% of the Cmax of an orally administered solution comprising Compound I.

In an embodiment of the third aspect of the invention, there is disclosed the method wherein the Cmax of the formulation is at least or greater than about 30% of the Cmax of an orally administered solution.

In another embodiment of the third aspect of the invention, there is disclosed the method wherein the Cmax of the formulation is at least or greater than about 35% of the Cmax of an orally administered solution.

In another embodiment of the third aspect of the invention, there is disclosed the method wherein the Tmax is at least about 3 hours.

In another embodiment of the third aspect of the invention, there is disclosed the method wherein the formulation is a tablet.

In another embodiment of the third aspect of the invention, there is disclosed the method wherein the formulation is a wet granulated tablet.

In a fourth aspect of the invention, there is disclosed an oral solid dosage formulation of Compound I of the formula

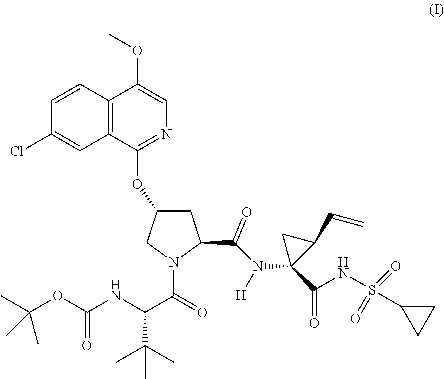 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

which comprises the active pharmaceutical ingredient and optionally one or more bioavailability enhancer/solubilizer, filler, binder, surfactant, disintegrant, glidant and/or lubricant.

In an embodiment of the fourth aspect of the invention, there is disclosed the formulation wherein the active pharmaceutical ingredient (API) (Compound I) is included in the range of 30-80% w/w, the filler is included in the range of 15-65% w/w, the binder is included in the range of 0-10% w/w, the disintegrant is included in the range of 1-20% w/w, the surfactant is included in the range of 2-10%, the glidant is included in the range of 0-10%, the bioavailability enhancer is included in the range of 2-20% and the lubricant is included in the range of 0.25-2.0% w/w.

In another embodiment of the fourth aspect of the invention, there is disclosed the formulation wherein the API is included in the range of 40-70% w/w, the filler is included in the range of 20-50% w/w, the binder is included in the range of 0-5% w/w, the disintegrant is included in the range of 5-15% w/w, the surfactant is included in the range of 3-6%, the glidant is included in the range of 0-5%, the bioavailability enhancer is included in the range of 4-6% and the lubricant is included in the range of 0.35-1.0% w/w.

In another embodiment of the fourth aspect of the invention, there is disclosed the formulation wherein the API is included in about 50% w/w, the filler is included in about 25-35% w/w, the binder is included in the range of 0-2% w/w, the disintegrant is included in the range of 10-12% w/w, the surfactant is included in about 4%, the glidant is included in the range of 0-3%, the bioavailability enhancer is included in about 4.67% and the lubricant is included in about 0.5% w/w.

In another embodiment of the fourth aspect of the invention, there is disclosed the formulation wherein the fillers and disintegrants are intragranular and extragranular.

In another embodiment of the fourth aspect of the invention, there is disclosed the formulation wherein the fillers are selected from lactose monohydrate and microcrystalline cellulose.

In another embodiment of the fourth aspect of the invention, there is disclosed the formulation wherein the binder is hydroxypropyl cellulose (HPC), polyvinyl pyrrolidone (PVP), starch or hydroxypropyl methylcellulose (HPMC).

In another embodiment of the fourth aspect of the invention, there is disclosed the formulation wherein the disintegrants are selected from croscarmellose sodium, crospovidone, starch and sodium starch glycolate.

In another embodiment of the fourth aspect of the invention, there is disclosed the formulation wherein the lubricant is magnesium stearate or sodium stearyl fumarate.

In another embodiment of the fourth aspect of the invention, there is disclosed the formulation wherein the glidant is colloidal silicon dioxide or silicon dioxide.

In another embodiment of the fourth aspect of the invention, there is disclosed the formulation wherein the surfactants is poloxamer, Polysorbate 80 or sodium lauryl sulfate.

In another embodiment of the fourth aspect of the invention, there is disclosed the formulation wherein the bioavailability enhancer/solubilizer is Vitamin E TPGS.

In another embodiment of the fourth aspect of the invention, there is disclosed the formulation wherein the API is Asunaprevir, the fillers are microcrystalline cellulose and/or lactose monohydrate, the binder is hydroxypropyl cellulose and/or PVP, the disintegrant is croscarmellose sodium, the surfactant is poloxamer and/or sodium lauryl sulfate, the bioavailability enhancer/solubilizer is Vitamin E TPGS, the glidant is colloidal silicon dioxide and the lubricant is magnesium stearate.

In a fifth aspect of the invention, there is disclosed an oral solid dosage formulation of Compound I of the formula

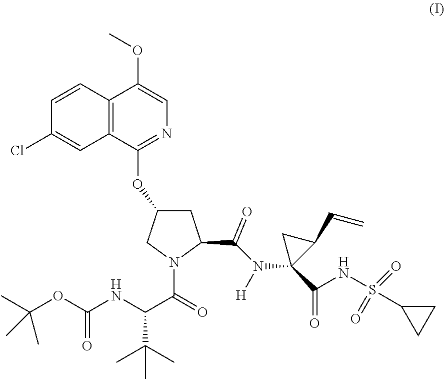 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

prepared by a wet granulation process which comprises the API and optionally one or more bioavailability enhancer, filler, binder, disintegrant, surfactant, glidant and/or lubricants.

In an embodiment of the fifth aspect of the invention, there is disclosed the oral solid dosage formulation prepared by a wet granulation process wherein the API is included in about 50% w/w, the filler is included in about 25-35% w/w, the binder is included in the range of 0-2% w/w, the disintegrant is included in the range of 10-12% w/w, the surfactant is included in about 4%, the glidant is included in the range of 0-3%, the bioavailability enhancer is included in about 4.67% and the lubricant is included in about 0.5% w/w.

In a sixth aspect of the invention, there is disclosed a method of administering an oral solid dosage formulation comprising orally administering to a fasted mammalian subject the formulation comprising Compound I having the formula

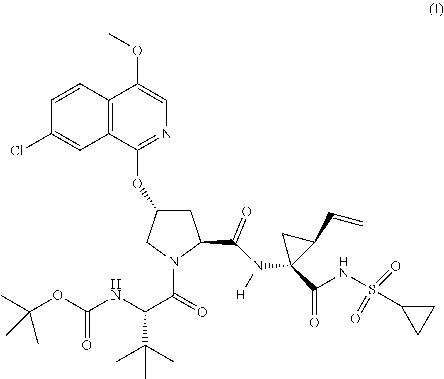 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

to provide a total blood plasma concentration profile of Compound I as measured by AUC at 24 hours after an initial dose of the formulation that is at least greater than about 15% of the total blood plasma concentration as measured by AUC at 24 hours of an initial dose of an orally administered solution comprising Compound I.

In an embodiment of the sixth aspect of the invention, there is disclosed the method wherein the AUC is at least greater than about 20% of the AUC at 24 hours of the solution when orally administered regardless if the subject is fasted or fed.

In another embodiment of the sixth aspect of the invention, there is disclosed the method wherein the AUC is at least greater than about 25% of the AUC at 24 hours of the solution when orally administered regardless if the subject is fasted or fed.

In another embodiment of the sixth aspect of the invention, there is disclosed the method wherein the formulation is a tablet.

In another embodiment of the sixth aspect of the invention, there is disclosed the method wherein the formulation is a wet granulated tablet.

In another embodiment of the sixth aspect of the invention, there is disclosed the method wherein the formulation comprises Vitamin E TPGS.

In another embodiment of the sixth aspect of the invention, there is disclosed the method wherein the formulation comprises at least 3% by weight Vitamin E TPGS.

In another embodiment of the sixth aspect of the invention, there is disclosed the method wherein the formulation comprises at least 4% by weight Vitamin E TPGS.

In another embodiment of the sixth aspect of the invention, there is disclosed the method wherein the formulation comprises at least 5% by weight Vitamin E TPGS.

In another embodiment of the sixth aspect of the invention, there is disclosed the method wherein the formulation comprises Vitamin E TPGS and at least one surfactant.

In another embodiment of the sixth aspect of the invention, there is disclosed the method wherein the formulation comprises Vitamin E TPGS and at least one surfactant selected from the group consisting of poloxamer and sodium lauryl sulfate.

In a seventh aspect of the invention, there is disclosed a method of administering an oral solid dosage formulation comprising orally administering to a fasted mammalian subject the formulation comprising at least one poorly soluble active pharmaceutical agent to provide a total blood plasma concentration profile as measured by AUC at 24 hours after an initial dose of the formulation that is at least greater than about 15% of the total blood plasma concentration as measured by AUC at 24 hours of an initial dose of an orally administered solution comprising the at least one active pharmaceutical agent.

In an embodiment of the seventh aspect of the invention, there is disclosed a method wherein the active pharmaceutical agent exhibits a significant food effect.

In another embodiment of the seventh aspect of the invention, there is disclosed a method wherein the at least one active pharmaceutical agent is included in the range from about 30 to about 80% w/w.

In another embodiment of the seventh aspect of the invention, there is disclosed a method wherein the formulation further comprises a bioavailability enhancer is included in the range from about 2 to about 20% and optionally comprises a surfactant in the range from about 2 to about 10%.

In another aspect of the invention, there is disclosed a method of treating an HCV infection, comprising the step of administering to a subject in need thereof an effective amount of the oral solid dosage formulation of the invention.

In another aspect of the invention, there is disclosed a method of inhibiting the HCV virus, comprising the step of administering to a subject in need thereof an effective amount of the oral solid dosage formulation of the invention.

In another aspect of the invention, there is disclosed a method of treating an HCV infection, comprising the step of administering to a subject in need thereof an effective amount of the oral solid dosage formulation prepared by a wet granulation process.

In another aspect, there is provided a method for treating an HCV infection, comprising administering to a patient in need thereof a therapeutically effective amount of a formulation of the present invention.

In another aspect, there is provided a method for preparing the formulation of the invention using a wet granulation process.

In another aspect, there is provided the use of the formulation of the present invention in therapy.

In another aspect, there is provided the use of the formulation of the present invention in the preparation of a medicament for the treatment of the hepatitis C virus.

In another aspect, there is provided a high drug load formulation of Compound I which mitigates food effects shown in other formulations comprising Compound I.

The invention may be embodied in other specific forms without departing from the spirit or essential attributes thereof. This invention also encompasses all combinations of preferred aspects and examples of the invention noted herein. It is understood that any and all aspects of the present invention may be taken in conjunction with any other embodiment to describe additional even more preferred embodiments of the present invention. Furthermore, any elements of an embodiment are meant to be combined with any and all other elements from any of the embodiments to describe additional embodiments.

### DEFINITIONS

“Therapeutically effective amount” is intended to include an amount of a compound of the present invention alone or an amount of the combination of compounds claimed or an amount of a compound of the present invention in combination with other active ingredients effective to inhibit or effective to treat or prevent HCV infection.

As used herein, the terms “treating” or “treatment” refer to the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting it development; and/or (c) relieving the disease-state, i.e., causing regression of the disease state.

As used herein, the term active pharmaceutical ingredient (API) or pharmaceutical agent refers to Asunaprevir (Compound I).

As used herein, the term “filler” refers to any pharmaceutically acceptable inert material or composition added to a formulation to add bulk. Suitable filler include for example, lactose monohydrate and microcrystalline cellulose.

As used herein, the term “disintegrant” refers to materials added to the composition to help it break apart and release the medicaments. Examples of disintegrants include, but are not limited to, non-saccharide water soluble polymers, such as cross-linked povidone. Other disintegrants that can be used include, for example, croscarmellose sodium, starch and sodium starch glycolate.

As used herein, the term “lubricant” refers to any pharmaceutically acceptable agent which reduces surface friction, lubricates the surface of the granule, and decreases the tendency to build up static electricity. Lubricants can also play a related role in improving the compression process by reducing the tendency of the material to adhere to the surface of compression tools. Thus, lubricants can serve as anti-adherents. Examples of suitable lubricants are magnesium stearate, stearic acid or other hydrogenated vegetable oil or triglycerides.

As used herein, the term “binder” refers to any pharmaceutically acceptable compound or composition that can help bind primary powder particles into agglomerates. Examples of suitable binding agents include, but are not limited to, hydroxypropyl cellulose (HPC), polyvinyl pyrrolidone (PVP), starch or hydroxypropyl methylcellulose (HPMC).

As used herein, the term “surfactant” refers to any pharmaceutically acceptable agent short for “surface acting agent” acted to modify a material or materials' physical or chemical properties such as wetability, solubility, stability, and miscibility.

As used herein, the term “bioavailability enhancer” refers to any pharmaceutically acceptable agent which is used to increase the absorption or solubility of a drug leading to an increase in bioavailability.

As used herein, the term “solubilizer” refers to any pharmaceutically acceptable agent which increases the solubility of a drug in a particular solvent or solution.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art. Such modifications fall within the scope of the appended claims.

Asunaprevir can be prepared following the procedure described in U.S. Pat. No. 6,995,174, issued Feb. 7, 2006.

The manufacturing process of this formulation generally involves mixing the drug with dry powder excipients, such as with one or more binder, disintegrant, and filler. Water is then added to this premix while it is being continuously mixed in a mixer such as a high shear granulator, fluid bed granulator or other manufacturing process where a solution is used for granulating a product. Alternatively, the binder may not be added as a dry powder to the premix, but dissolved in water. Addition of water with continuous mixing leads to the formation of granules, or granulation. The granules are then dried in dry, hot air using equipment and processes such as fluid bed drying or tray drying. Once dried, the granules may be milled using equipment such as Comill or Fitzmill to reduce and/or reduce the width of distribution of particle size of granules. The granules are then mixed with extra-granular ingredients, which may include optionally one or more of disintegrant, filler, glidant, and lubricant. The final blend is then compressed on a tablet press into core tablets. The core tablets may optionally be coated with a nonfunctional film coat.

Further examples of formulating techniques can be found in Pandey et al., Pharmaceutical Development and Technology, 2013; 18(1): 296-304.

### Example 1

### Wet Granulation

The following is a typical procedure for manufacturing the wet granulated solid formulation of the invention.

Vitamin E TPGS is dissolved into water, by adding up to 20% w/w solution Vitamin E TPGS to water while agitating. The API (prepared as described in U.S. Pat. No. 6,995,174), filler, surfactant/solubilizer (e.g., poloxamer), disintegrant (e.g., croscarmellose sodium), and binder (e.g., polyvinylpyrrolidone) if present, are blended together. The granulation is performed by adding the solution of Vitamin E TPGS to the dry blend, while being mixed in the high shear granulator.

Wet granulations are removed from the granulator, and screened as needed. The granulation is dried in a fluid bed, oven or other type of drier. The dried granulation is milled or screened as needed. The dried granulation may be mixed with one or more of the following materials, glidant(s) (e.g., colloidal silicon dioxide), disintegrant(s) (e.g., croscarmellose sodium), lubricant(s) (e.g., magnesium stearate), and/or filler(s) (e.g., microcrystalline cellulose) to complete the formulation.

The following 5 Tables disclose typical formulations prepared using the above procedure.

TABLE 1

| Ingredient | mg/tablet | % (w/w) |
|---|---|---|
| Compound I | 200 | 50 |
| Lactose Monohydrate | 60 | 16 |
| Microcrystalline Cellulose | 58 | 14.5 |
| Croscarmellose Sodium | 46 | 11.5 |
| Vitamin E TPGS | 18 | 4.5 |
| Poloxamer | 16 | 4 |
| Magnesium Stearate | 2 | 0.5 |
| Total | 400 | 100 |

TABLE 2

| Ingredient | mg/tablet | % (w/w) |
|---|---|---|
| Compound I | 200 | 50 |
| Lactose Monohydrate | 56.76 | 14.19 |
| Microcrystalline Cellulose | 60.56 | 15.14 |
| Croscarmellose Sodium | 46 | 11.5 |
| Vitamin E TPGS | 18.68 | 4.67 |
| Sodium Lauryl Sulfate | 16 | 4 |
| Magnesium Stearate | 2 | 0.5 |
| Total | 400 | 100 |

TABLE 3

| Ingredient | mg/tablet | % (w/w) |
|---|---|---|
| Compound I | 200 | 50 |
| Lactose Monohydrate | 52.76 | 13.19 |
| Microcrystalline Cellulose | 56.56 | 14.14 |
| Croscarmellose Sodium | 46 | 11.5 |
| Vitamin E TPGS | 18.68 | 4.67 |
| Sodium Lauryl Sulfate | 16 | 4 |
| Polyvinylpyrrolidone | 8 | 2 |
| Magnesium Stearate | 2 | 0.5 |
| Total | 400 | 100 |

TABLE 4

| Ingredient | mg/tablet | % (w/w) |
|---|---|---|
| Compound I | 200 | 50 |
| Lactose Monohydrate | 52.76 | 13.19 |
| Microcrystalline Cellulose | 56.56 | 14.14 |
| Croscarmellose Sodium | 46 | 11.5 |
| Vitamin E TPGS | 18.68 | 4.67 |
| Sodium Lauryl Sulfate | 16 | 4 |
| Hydroxypropyl cellulose | 8 | 2 |
| Magnesium Stearate | 2 | 0.5 |
| Total | 400 | 100 |

TABLE 5

| Ingredient | mg/tablet | % (w/w) |
|---|---|---|
| Compound I | 200 | 50 |
| Lactose Monohydrate | 64 | 16 |
| Microcrystalline Cellulose | 64 | 16 |
| Croscarmellose Sodium | 46 | 11.5 |
| Poloxamer | 16 | 4 |
| Polyvinylpyrrolidone | 8 | 2 |
| Magnesium Stearate | 2 | 0.5 |
| Total | 400 | 100 |

### Example 2

### Bioequivalence Study

A study was conducted to compare conventional tablet dosage forms. The goal of the study was to assess the in vivo performance of various prototypes of conventional tablet dosage forms (wet and dry granulated tablets) in a dog model.

The following five formulations were tested:

1. Phase 2 Clinical (dry granulated (DG)) tablet 2×200 mg.

2. Wet granulated (WG) Vitamin E TPGS/poloxamer tablet: 50% w/w Compound I+4.67% w/w Vitamin E TPGS+4% w/w poloxamer+14.33% w/w microcrystalline cellulose+15% lactose monohydrate+11.5% w/w croscarmellose sodium+0.5% w/w magnesium stearate; 2×200 mg (Corresponds to the formulation shown above in Table 1).

3. WG poloxamer tablet: 50% w/w Compound I+2% w/w polyvinyl pyrrolidone+4% w/w poloxamer+16% microcrystalline cellulose+16% w/w lactose monohydrate+11.5% w/w croscarmellose sodium+0.5% w/w magnesium stearate; 2×200 mg (Corresponds to the formulation shown above in Table 5).

4. Amorphous API tablet: 50% w/w Compound I+41.25% w/w microcrystalline cellulose+5% w/w croscarmellose sodium+2% w/w silicon dioxide+1% w/w sodium lauryl sulphate+0.75% w/w magnesium stearate; 2×200 mg.

5. Solution Formulation: Polyethylene glycol 400:EtOH: polysorbate 80 (50:35:15), 20 mg/mL.

The protocol used was as follows:

Crossover in 4 fasted, pentagastrin-treated, male dogs (˜10 kg), 400 mg dose flushed with 50 ml water. For food-effect studies in the fed state, dogs were given 50 ml high-fat meal substitute instead of water.

The following was observed (as shown in Table 6 below):

TABLE 6
PK Parameters for Wet(WG) and Dry Granulated (DG) Formulations in a Dog Model

| | | | | | % CV | | | | Relative | | |
|---|---|---|---|---|---|---|---|---|---|---|---|
| | | Cmax | | Relative | % Std | Tmax | AUC | | BA | | |
| | | Mean | SD | Cmax | Dev | median | Mean | SD | Mean | SD | % CV |
| DG tablet | fasted | 8693.61 | 585.82 | 20.16 | | 2.50 | 40519.42 | 8795.62 | 8.04 | 1.75 | 21.71 |
| WG TPGS/Pluronic | fasted | 14381.70 | 3776.03 | 33.35 | 26.26 | 3.00 | 104507.16 | 28685.28 | 20.73 | 5.69 | 27.45 |
| WG Pluronic | fasted | 8944.45 | 6743.93 | 20.74 | 75.40 | 3.00 | 45711.73 | 38370.23 | 9.07 | 7.61 | 83.94 |

Tablet formulations in the fasted state range from 8-21% bioavailability.
The Vitamin E TPGS/poloxamer (Pluronic) wet granulation tablet is the best performing tablet with the highest Cmax and AUC compared to the other prototypes.
The presence of poloxamer alone does not improve performance.
The presence of Vitamin E TPGS as a solubilizer improves performance, and also significantly reduces variability compared to the wet granulated tablet without Vitamin E TPGS.

The dog model showed that Compound I has a significant positive food effect. The effect is related to low API dissolution/high solubility in lipid environment and slower GI motility (higher Tmax). It was observed that the presence of Vitamin E TPGS in the tablet formulations appears to improve its in vivo performance and reduce variability compared to the wet granulated formulation that did not contain Vitamin E TPGS.

A clinical study in humans was conducted to evaluate the pharmacokinetics and effect of fasted vs. fed subjects on the oral absorption of various formulations of Compound I. Using the relative bioavailability and comparing the wet granulated (WG) tablet containing Vitamin E TPGS with a dry granulated (DG) tablet that had previously been used, the following results were observed:

| | Fed | Fasted | Ratio |
|---|---|---|---|
| DG Tablet | 43% | ~10% | 4.3 |
| WG Tablet | 34% | 26% | 1.31 |

Thus, the wet granulated formulation of the invention was found to provide consistent oral bioavailability of Compound I without regard for the dietary state of the patient.

Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

What is claimed is:

1. An oral solid dosage formulation comprising at least one pharmaceutical agent comprising Compound I of the formula 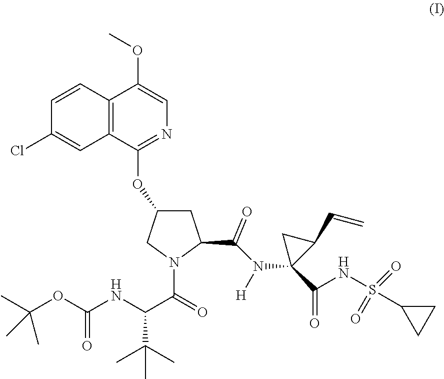 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]
in the range of 30-80% w/w and a bioavailability enhancer is included in the range of 2-20% w/w of the total formulation.

2. The formulation of claim 1, wherein a surfactant is included in the range of 2-10%.

3. The formulation of claim 1, wherein the active pharmaceutical agent is included in the formulation in an amount of at least about 40% w/w.

4. The formulation of claim 1, wherein the active pharmaceutical agent is included in the formulation in an amount of at least about 50% w/w.

5. The formulation of claim 1, wherein the active pharmaceutical agent is included in the formulation in an amount of at least about 60% w/w.

6. A method of administering an oral solid dosage formulation comprising orally administering to a fasted mammalian subject the formulation comprising Compound I having the formula 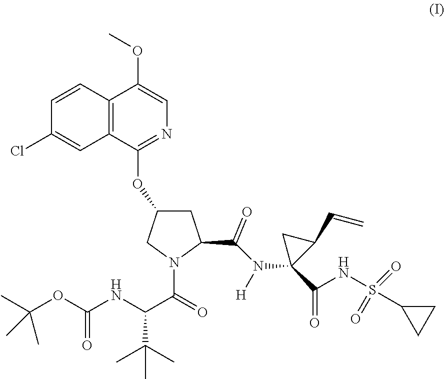 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]
to provide a total blood plasma concentration profile of Compound I as measured by AUC at 24 hours after an initial dose of the formulation that is at least greater than about 15% of the total blood plasma concentration as measured by AUC at 24 hours of an initial dose of an orally administered solution comprising Compound I.

7. The method of claim 6, wherein the AUC is at least greater than about 20% of the AUC at 24 hours of the solution when orally administered regardless if the subject is fasted or fed.

8. The method of claim 6, wherein the AUC is at least greater than about 25% of the AUC at 24 hours of the solution when orally administered regardless if the subject is fasted or fed.

9. The method of claim 6, wherein the formulation comprises Vitamin E TPGS.

10. The method of claim 9, wherein the formulation comprises at least 4% by weight Vitamin E TPGS.

11. The method of claim 10, wherein the formulation comprises Vitamin E TPGS and at least one surfactant.

12. The method of claim 11, wherein the formulation comprises Vitamin E TPGS and at least one surfactant selected from the group consisting of poloxamer, sodium lauryl sulfate and Polysorbate 80.

13. A wet granulated tablet formulation comprising at least one pharmaceutical agent comprising Compound I of the formula 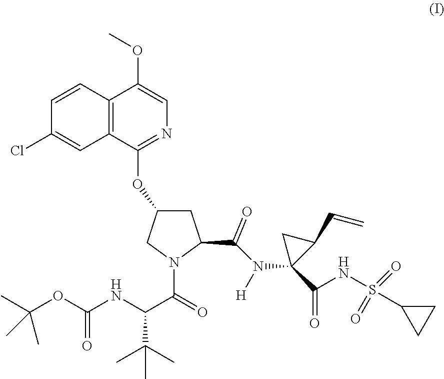 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]
in the range of 30-80% w/w and a bioavailability enhancer is included in the range of 2-20% w/w of the total formulation.

14. The formulation of claim 13, wherein a surfactant is included in the range of 2-10%.

15. The formulation of claim 13, wherein the active pharmaceutical agent is included in the formulation in an amount of at least about 50% w/w.

16. A method of administering a formulation comprising orally administering to a fasted mammalian subject the formulation comprising Compound (I) having the formula 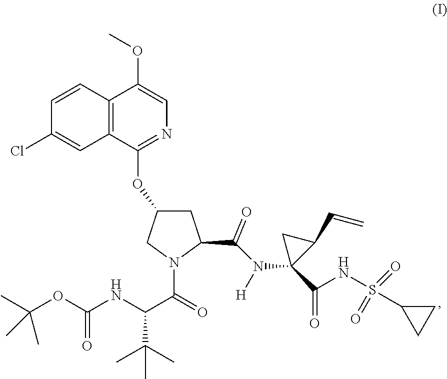 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]
and has a fed to fasted ratio lower than at least about 2.0.

17. The method according to claim 16, wherein the fed to fasted ratio is less than about 1.75.

18. The method according to claim 16, wherein the fed to fasted ratio is less than about 1.50.

19. The method according to claim 16, wherein the fed to fasted ratio is less than about 1.20.

20. A method of treating an HCV infection whereby any food effect of the formulation are mitigated, comprising the step of administering to a subject in need thereof a therapeutically effective amount of the formulation of claim 1.

21. A method of treating an HCV infection whereby any food effects of the formulation are mitigated, comprising the step of administering to a subject in need thereof a therapeutically effective amount of the formulation of claim 13.
